(19) United States
(12) Patent Application Publication
(10) Pub. No.: US 2005/0238671 A1
(43) Pub. Date: Oct. 27, 2005

(54) Superantigen enhancement of specific immune responses

(76) Inventors: Howard M. Johnson, Gainesville, FL (US); Barbara A. Torres, Gainesville, FL (US); Scott L. Kominsky, Columbia, MD (US); Amy C. Hobeika, (US)

Correspondence Address: BEUSSE BROWNLEE WOLTER MORA & MAIRE, 390 N. ORANGE AVENUE, SUITE 2500, ORLANDO, FL, 32801, US

(21) Appl. No.: 11/147,046

(22) Filed: Jun. 7, 2005

Related U.S. Application Data
(63) Continuation of application No. 09/808,718, filed on Mar. 15, 2001.
(60) Provisional application No. 60/189,346, filed on Mar. 14, 2000.
(60) Provisional application No. 60/194,951, filed on Apr. 1, 2000.

(51) Int. Cl. A61K 39/00

(52) U.S. Cl. 424/277.1

(57) ABSTRACT

The invention relates to superantigen mediated expansion of antigen-specific T cells for cancer and infectious agent treatment/prophylaxis. Mice were injected with inactivated B16F10 mouse melanoma cells, followed by injection with a combined SEA/SEB injection or a sham injection on days 3 and 6, followed by day 4 challenge with live melanoma cells. The SEA/SEB recipient mice survived longer post-challenge and had a higher CTLs against tumor cells than did the sham injected mice. SEA/SEB TCR activation has been reported to be independent of antigen specificity of TCRs. This invention provides a method whereby a combination of Staphylococcal enterotoxin superantigens is used to enhance specific immune responses to activating antigens to enhance immune responses against cancers and infectious agents.

### RELATED APPLICATIONS

This is a continuation of U.S. Ser. No. 09/808,718 filed Mar. 15, 2001, which in turn claims the benefit of U.S. Ser. No. 60/189,346 filed Mar. 14, 2000, and U.S. Ser. No. 60/194,951 filed Apr. 1, 2000.

The research which forms the basis of this patent disclosure was supported in part by NIH Grant Number R37 AI25904. Accordingly, the U.S. Government may have certain rights in this invention.

### FIELD OF THE INVENTION

This invention is directed to methods and compositions useful in enhancement of specific immune responses, including those against pathogens and tumor antigens, by optimized use superantigens.

### BACKGROUND OF THIS INVENTION

The term “superantigen” as used herein is to be understood as meaning any substance which, while preferably is ineffective at inducing an immune response against itself, is effective in enhancing an immune response to another specific antigen.

Superantigens, as defined herein, have been known in the art for some time. Superantigen microbial proteins that are potent activators of large numbers of CD4⁺ T cells interact with MHC class II molecules on antigen-presenting cells and with the variable region of the beta chain (Vβ) of the T cell receptor (TCR) on T cells, thereby causing profound T cell activation (1). In this way, as many as 20% of the T cells in a typical T cell population can be activated by the superantigen. This invention provides a method for use of superantigens to expand large numbers of T cells to treat or prevent a wide variety of pathologic conditions, including various forms of cancer, specifically in cancers where tumor-specific antigens can be used to confer at least some protection to the host. This invention disclosure demonstrates our discovery that administration of superantigens after immunization with killed melanoma cells induces significantly longer survival times for mice challenged with live melanoma cells (50% of mice are protected against lethal doses of melanoma cells, while 50% of mice treated with vaccine alone or superantigen alone die within about 31-38 days).

### BRIEF DESCRIPTION OF THE DRAWINGS

FIG. 1 shows data comparing the number of days mice survive a melanoma challenge when vaccinated using various immunization protocols, including first immunizing the mice with the tumor antigen, followed by administration of superantigen.

FIG. 2 shows data comparing the timing of superantigen administration following antigen administration to the timing used in FIG. 1.

FIG. 3 shows data in which the cytolytic effect of spleen cells activated by administration of tumor antigen followed by superantigen administration.

FIG. 4 shows ELISA data showing development of humoral immune responses as a result of antigen administration in combination with subsequent administration of superantigen, as compared with when antigen or superantigen is administered alone. For the BSA+Superantigen experiment, the protocol employed administration of 0.5 mg of BSA intraperitoneally on day 0; administration of SEA+SEB (25 micrograms each) intraperitoneally on day 7; harvesting of blood samples on day 14.

FIG. 5 shows the inhibitory effect of the cytokine IFNγ, the production of which is enhanced by administration of superantigen, on growth of melanoma cells.

FIG. 6 shows protection of mice against tumor challenge is dependent on the timing of superantigen administration following vaccination. C57BL/6 mice were vaccinated i.p with 1×10⁶ irradiated B16F10 melanoma cells at day 0 and subsequently injected i.p. with 25 μg each SEA and SEB at either day 6 or 10. As can be seen, Mice receiving no treatment, vaccination only, and SEA/SEB only served as controls. All mice were challenged i.p. with 2.5×10⁵ live B16F10 cells at day 13. Mice were evaluated on a daily basis and sacrificed when moribund.

FIG. 7 shows protection of mice against tumor challenge is dependent on dose of superantigen administered following vaccination. C57Bl/6 mice were vaccinated i.p. with 1×10⁶ irradiated B16F10 melanoma cells at day 0 and subsequently injected i.p. with 6.25 μg, 12.5 μg ,or 25 μg each SEA and SEB at day 10. As can be seen, Mice receiving no treatment, vaccination only, and SEA/SEB only served as controls. All mice were challenged i.p. with 2.5×10⁵ live B16F10 cells at day 13. Mice were evaluated on a daily basis and sacrificed when moribund.

FIG. 8 shows vaccination followed by superantigen adminsitration results in increased tumoricidal activity. C57Bl/6 mice were vaccinated i.p. with 1×10⁶ irradiated B16F10 melanoma cells at day 0 and subsequently injected i.p. with 25 μg each SEA and SEB at day 6. As can be seen, Mice receiving vaccination only, and SEA/SEB only served as controls. Splenocytes were harvested at day 9 and CTL activity was determined by standard 4 hour 51Cr release assay.

FIG. 9 shows superantigen administration results in increased serum levels of IFNγ. C57BL/6 mice were injected i.p. with 25 μg each SEA and SEB. Serum samples were subsequently collected at appropriate time points and analyzed for IFNγ by ELISA. No IFNγ was detected in serum from mice receiving no treatment. (43 μg is approximately equal to 1 unit IFNγ.).

FIG. 10 shows that IFNγ inhibits cellular proliferation of B16F10 melanoma cells in a dose-dependent manner in vitro. Cells (1×10⁴ cells/well) were treated with or without IFNγ at concentrations ranging from 0.1 to 10,000 units/ml for 72 hours. Cells were then harvested and the total number of live cells was determined by direct cell count. Cell viability was approximately 88%. Data from representative experiments are expressed as total number of cells per sample .+-.S.D. Statistical significance was shown by Student's t-test between the number of cells in the presence and absence of IFNγ (P<0.04).

FIG. 11 shows protection of long term survivors against rechallenge with live B16F10 melanoma cells. C57BL/6 mice surviving beyond 150 days following vaccination, superantigen agministration, and subsequent live B16F10 tumor challenge were rechallenged i.p with 1×10⁴ live B16F10 cells at day 0. Mice receiving no treatment served as control. Mice were evaluated on a daily basis and sacrificed when moribund.

FIG. 12 shows vaccination followed by superantigen administration induces a specific immune response. C57BL/6 mice were vaccinated i.p. with 1×10⁶ irradiated B16F10 melanoma cells at day 0 and subsequently injected i.p. with 25 μg each SEA and SEB at day. 10. Mice receiving no treatment, vaccination only, and SEA/SEB only served as controls. All mice were challenged i.p. with 1.times. 10⁶ live Lewis lung carcinoma (LL/2) cells at day 13. Mice were evaluated on a daily basis and sacrificed when moribund.

FIG. 13 shows superantigen prolongs the survival of mice with established tumor. C57BL/6 mice were challenged i.p. with live B16F10 cells at day 0 and subsequently injected i.p with 25 μg each SEA and SEB at day 6. Mice receiving no treatment served as control. Mice were evaluated on a daily basis and sacrificed when moribund.

### SUMMARY OF THE INVENTION

This invention relates to superantigen mediated expansion of antigen-specific T cells for cancer and infectious agent treatment/prophylaxis. Mice were injected with inactivated B16F10 mouse melanoma cells, followed by injection with a combined SEA/SEB injection or a sham injection on days 3 and 6, followed by day 4 challenge with live melanoma cells. The SEA/SEB recipient mice survived longer post-challenge and had higher CTLs against tumor cells than did the sham injected mice. SEA/SEB TCR activation has been reported to be independent of antigen specificity of TCRs. This invention provides a method whereby a combination of Staphylococcal enterotoxin superantigens is used to enhance specific immune responses to activating antigens to enhance immune responses against cancers and infectious agents.

Accordingly, it is one object of this invention to provide superantigen compositions with varying VP specificities for enhancing antigen-specific immune responses to various pathologic conditions associated with specific antigenic mediators or markers, including but not limited to tumor associated antigens.

A further object of this invention is to provide different combinations of superantigens in order to expand the Vβ repertoire against specific antigens, including but not limited to tumor associated antigens.

A further object of this invention is to provide a method for inducing cellular, humoral and cytokine responses that confer host defenses against various antigens associated with a wide range of pathologic conditions, including but not limited to tumor associated antigens.

A further object of this invention is to provide a method of vaccination against various pathologic conditions, including infectious diseases, tumors and the like which comprises administration of specific antigens associated with the specific pathologic condition sought to be prevented, followed by a regimen of booster vaccinations and superantigen administration at optimized times and dosages, in relation to the timing and dosage of administered vaccine.

Further objects and advantages of this invention will be appreciated from a review of the complete disclosure provided herein, including the claims appended hereto.

### DETAILED DISCLOSURE

Microbial superantigens are potent activators of CD4⁺ T cells. Superantigens can activate large number of CD4⁺ T cells, thereby causing the production of high levels of cytokines (1). The predominant cytokines produced and released during superantigen activation are interleukin-2 (IL-2) and gamma interferon (IFNγ) both of which are intimately involved in the cascade of cytokines produced during immune responses. The levels of cytokines produced are higher than those normally achieved during conventional antigen-induced T cell activation, presumably due to the potency of superantigens in activating large numbers of cells.

Superantigens interact with T cells in a manner that significantly differs from conventional antigens. Superantigens function as intact molecules and bind directly to MHC class II molecules on the surface of antigen-presenting cells (2). Superantigens can be presented to T cells by many types of immunologic class II-bearing cells, including monocytes/macrophages, B cells, and natural killer cells (3). Binding to class II occurs at a site outside the antigen-binding groove (4, 5). This complex of superantigen/MHC class II interacts directly with the Vβ region of the TCR on T cells, thereby causing T cell activation (6).

To date, approximately 60 different Vβ elements of human TCRs have been identified. The subsets of Vβ bearing T cells that are activated by one superantigen may differ from those activated by another superantigen (Table 1). Three toxins produced by Staphylococcus aureus can be used as examples. Toxic shock syndrome toxin-1 (TSST-1) interacts with mouse T cells bearing Vβ 15 and 16, whereas staphylococcal enterotoxin A (SEA) interacts with Vβ1, Vβ3, Vβ10, Vβ11, and Vβ17, and SEB activates T cells expressing Vβ7 and Vβ8.1-8.3 (7). Thus, superantigens induce expansions of unique subsets of Vβ specific T cells, as many as 20% of cells in a given T cell population.

T cell stimulation by superantigens causes proliferation and the prodigious production of cytokines, primarily from CD4⁺ cells (8-11). The predominant cytokines produced and released during superantigen activation are interleukin-2 (IL-2) and gamma interferon (IFNγ) both of which are intimately involved in the cascade of cytokines produced during immune responses. The levels of cytokines produced are higher than those normally achieved during conventional antigen-induced T cell activation, presumably due to potency of superantigens in activating large numbers of cells.

Upon stimulation by superantigens, nave T cells respond and then quickly become anergized and/or deleted (12, 13). On the other hand, T cells that are actively undergoing activation by specific antigen at the time of superantigen stimulation do not become anergized (14). This is an important characteristic of superantigens that can be exploited when attempting to enhance specific antigen responses. Superantigens can cause anergy and/or deletion of potentially competing T cells bearing the same Vβ element(s) as T cells of a desired antigen specificity. In other words, T cells with a desired antigen specificity will be further and more potently expanded by superantigens while other T cells will become anergized. Thus there would be less “competition” for cytokines and the specific immune response will be amplified.

Superantigens have been implicated in autoimmune disorders, including experimental allergic encephalomyelitis (EAE), the mouse model for multiple sclerosis. EAE involves both the cellular and humoral arms of the immune response in that mice with EAE have autoreactive T cells and antibody production to the autoantigen, myelin basic protein (15). Superantigens have been shown to exacerbate EAE, suggesting that immune responses are enhanced by superantigens. This negative property of superantigens, and the complexity of the variously positive and negative results achieved through administration of superantigens has resulted in great trepidation when considering use of superantigens in the art of immunotherapy. The present invention is directed to methods and compositions whereby superantigen induced effects may be exploited to advantage in cases where immune responses are needed to be rapidly and potently enhanced, such as in the case of cancer and in the immunoprophylaxis of other specific antigen-associated diseases (e.g. AIDS, via enhancement of anti-HIV antigen immune responses, Hepatitis B, via enhancement of anti-hepatitis B virus core or surface antigen immune responses, and the like).

The present patent disclosure reveals positive immunoprophylactic effects of appropriately administered superantigens on cancer using a mouse melanoma model (16). In this model, mice are challenged with live B16F10 melanoma cells administered intraperitoneally. Tumor cells given in this manner usually results in the death of C57Bl/6 mice approximately 14 days after challenge. Disclosed herein are experiments in which timing of treatment with superantigens is optimized in relation to timing of tumor cell vaccination using this mouse model. These studies focus on administration of a combination of two superantigens, SEA and SEB. These superantigens were chosen for their wide arrays of Vβ specificities. However, based on the present disclosure, those skilled in the art are fully enabled to apply use of other superantigens and combinations thereof for enhancement of immune responses to any other identified antigen. Based on the present disclosure, those skilled in the art are enabled to develop specific superantigen compositions, including a single superantigen or combinations of superantigens, and portions thereof, for administration at specific times in relation to the time of administration of specific antigens, to induce an optimized cellular and humoral immune responses specific to the given antigen. Depending on the particular antigen used, from the present disclosure those skilled in the art will appreciate that a different combination of superantigens may be optimized, but that such optimization may be achieved through routine application of the methods and principles taught herein. To this end, the Vβ specificities of certain known superantigens are presented in Table 1:

TABLE 1
Vβ T Cell Specificities of Staphylococcal Superantigens

| | Vβ Specificity | |
|---|---|---|
| Superantigen (Source) | Mouse | Human |
| SEA (S. aureus) | 1, 3, 10, 11, 17 | 1.1, 5, 6's, 7.3-7.4, 9.1 |
| SEB (S. aureus) | 7, 8.1-8.3 | 3, 12, 14, 15, 17, 20 |
| SEC1 (S. aureus) | 3, 8.2, 8.3, 11 | 3, 6.4, 6.9, 12, 15 |
| SEC2 (S. aureus) | 3, 8.2, 10, 17 | 12, 13.2, 14, 15, 17, 20 |
| SEC3 (S. aureus) | 7, 8 | 3, 5, 12, 13.2 |
| SED (S. aureus) | 3, 11, 17 | 5, 12 |
| SEE (S. aureus) | 11, 15, 17 | 5.1, 6's, 8, 18 |
| TSST-1 (S. aureus) | 15, 16 | 2 |
| MAM | 5.1, 6, 8.1-8.3 | 3, 17 |

Based on the various Vβ specificities of different superantigens, those skilled in the art will appreciate that different cohorts of T-cell subsets may be expanded by judicious use of different combinations of superantigens. In this fashion, the enhancement of immune activation achieved by the superantigen composition used may be tailored to any specific antigen, to maximize the specific immunological response that is elicited. In addition, those skilled in the art will appreciate that it is frequently not necessary for the complete superantigen molecule to be used to elicit the desired superantigen effect. Thus, superantigen agonist and antagonist peptides have been identified which may be employed to advantage according to the methods disclosed herein. For this purpose, the disclosure of U.S. Pat. Nos. 5,859,207; 5,545,716; 5,519,114; 5,795,974; 5,968,514; 5,891,438; are hereby incorporated by reference. Thus, superantigen peptides disclosed in any of these patents may be used to advantage according to the methods of this invention to form compositions for enhancing the immune response induced by any specific antigen. Combinations of peptides with other peptides, as well as peptides with complete superantigen molecules are therefore contemplated by this invention.

Having generally described this invention, the following examples are provided to extend the written description of this invention. However, the scope of this invention should not be construed as being limited to the specifics of the examples which follow. Rather, the scope of the invention should be understood to be defined by the claims appended hereto.

### EXAMPLE 1

### Induction of Anti-Tumor Immune Responses, with and without Superantigen Administration

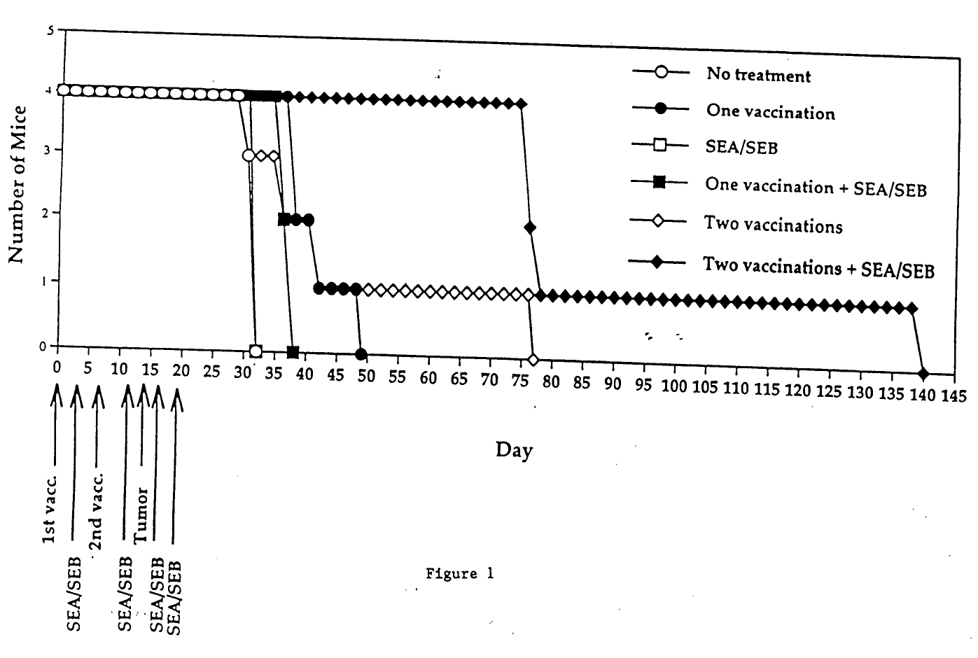

In one series of experiments, mice were given an intraperitoneal vaccination consisting of inactivated B16F10 mouse melanoma cells on Days 0 and 7, and intraperitoneal injections of a combination of SEA and SEB on Days 4, 11, 16 and 18. On Day 14, mice were challenged with live tumor cells and monitored thereafter (FIG. 1). Melanoma mice that received live intraperitoneal tumor cell challenge died by Day 32 of the experiment, as did mice that received only SEA/SEB (i.e., no vaccinations) or only inactivated tumor cell vaccination. The group of mice that received two vaccinations in concert with superantigens survived the longest—100% survival up to Day 76, with one mouse surviving until Day 140, ultimately succumbing to a recurrence of melanoma. Thus, superantigens significantly extended the survival times of mice challenged with live melanoma cells.

Mice that received either 1 or 2 vaccinations (in the absence of superantigens) survived slightly longer than controls, indicating the importance of vaccination. Further, we observed that mice that received a treatment consisting of one vaccination and superantigens died earlier than mice receiving one vaccination only. This can be explained by the ability of superantigens to anergize and/or delete naive T cells. It is likely that superantigen administration four days after vaccination caused significant anergy/deletion in a pool of T cells that were specific for the tumor cells but that had not had time to respond to the vaccine. The fact that these mice survived longer than controls suggests that some antigen-specific T cell activation did occur. This is further suggested by the survival of mice that received a second round of vaccination and superantigens, where the second round of treatment was able to expand those tumor-specific T cells to the extent where mice survived the longest. Thus, the timing of superantigen administration following vaccination is very important.

### EXAMPLE 2

### Significance of the Time of Superantigen Administration in Relation to the Timing of Antigen Administration for Enhancement of Anti-Antigen Specific Immune Responses

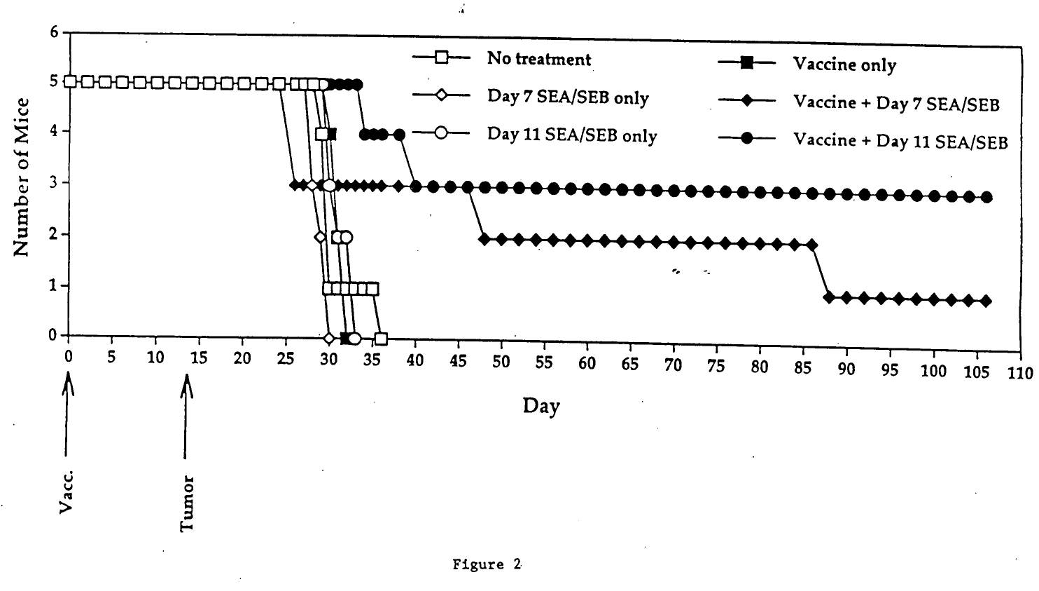

The results of the studies discussed above led us to study the timing of superantigen administration relative to inactivated tumor cell vaccination and superantigen administration. Mice were vaccinated once with inactivated tumor cells on Day 0 and superantigens were given either on Day 7 or Day 11. Note that previously the first superantigen treatment occurred at four days, which was found to be too early to achieve an optimal anti-tumor response. Mice were challenged on Day 14 with live melanoma cells. As can be seen in FIG. 2, three out of five mice (60%) that received the tumor cell vaccine plus SEA/SEB on Day 11 were still alive at 106 days. This is at least three times longer survival than controls, which died by Day 36. The protected mice did not show any signs of tumor recurrence. Similarly, mice that received vaccine on Day 0 and superantigen on Day 7 showed significantly longer survival times, with one mouse still alive at 106 days, demonstrating the value of this treatment methodology for treatment of melanoma. Those skilled in the art will appreciate that demonstration of efficacy in a mouse model is generally accepted as a valuable predictor of efficacy in other species, including humans.

### EXAMPLE 3

### Mechanism of Superantigen Enhancement of Antigen-Specific Immune Responses—Cell Mediated Responses

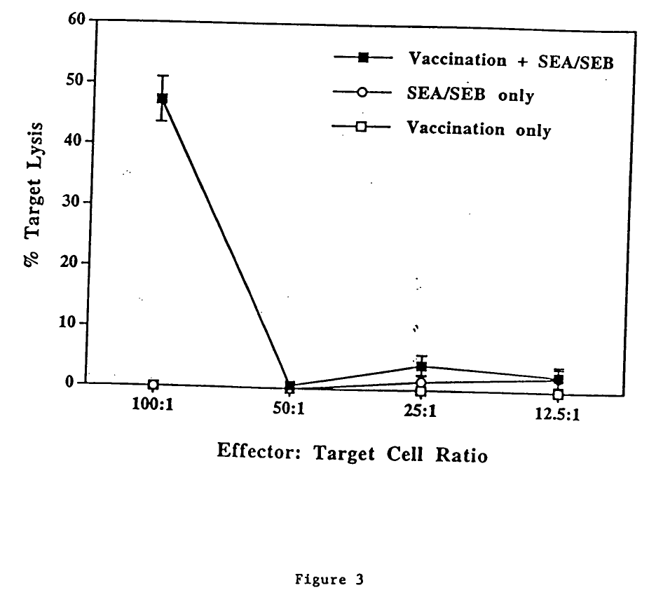

While not wishing to be bound by mechanistic considerations, in efforts at elucidation of the mechanism of action of superantigen, mice were vaccinated with inactivated tumor cells, subsequently given SEA/SEB, and spleen cells were harvested for cytotoxic T lymphocyte (CTL) activity. Spleen cells were incubated with inactivated melanoma B16F10 target cells that were labeled with ⁵¹Cr and target cell lysis was assessed 4 hours later. As seen in FIG. 3, significant lysis of target cells (48%) was achieved at an effector:target ration of 100:1. These data correlate with the protection observed in the death rate studies. Thus, significant CTL activity is observed in mice treated with a combination of vaccine and superantigens.

### EXAMPLE 4

### Mechanism of Superantigen Enhancement of Antigen-Specific Immune Responses—Humoral Immune Responses

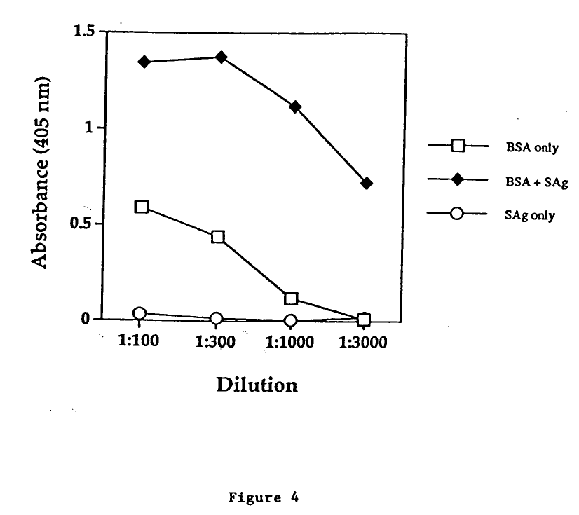

Superantigens are known to activate CD4⁺ T helper type 1 (T_{H1}) cells or inflammatory T cells, which are involved in cellular immune responses such as aiding in the generation of cytotoxic CD8⁺ T cells. We were interested in determining the superantigen effects on CD4⁺ T_{H2} cells, which act as helper cells for antibody production by B cells. Thus, in addition to determining the effects of superantigens on cellular immune responses to tumor cells, we undertook studies on the effects of superantigens on humoral immune responses. Specifically, we analyzed the effects of superantigen enhancement of antibody production to bovine serum albumin (BSA) in mice. C57Bl/6 mice were injected with BSA alone, BSA followed by SEA/SEB on Day 7, or SEA/SEB alone on Day 7. Serum levels of anti-BSA antibodies were determined by ELISA on Day 14. SEB enhanced the BSA antibody response by approximately 2.3-fold (FIG. 4). These studies have importance for enhancing the antibody response against soluble proteins, and to humoral responses to tumors and tumor associated and other antigens.

### EXAMPLE 5

### Mechanism of Superantigen Enhancement of Antigen-Specific Immune Responses—Cytokine Responses

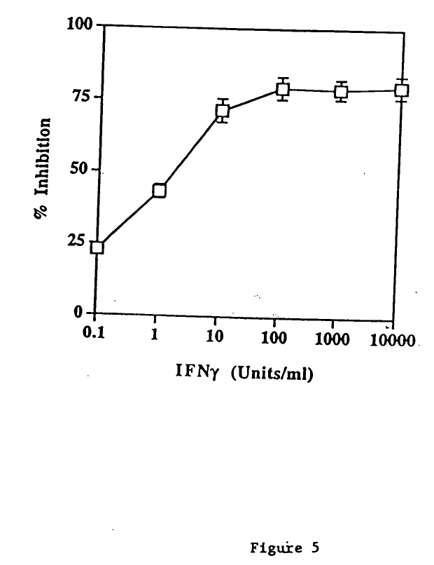

One of the hallmarks of superantigen activation of T cells is the production of cytokines, one of which is IFNγ. In addition to its important immunomodulatory activities, such as enhancement of CTL, natural killer, and macrophage tumoricidal activities, it is known to have direct anti-proliferative properties (17-19). Thus, we investigated the effects of IFNγ on the growth rate of B16F10 melanoma cells in an in vitro assay. Cells were plated in 24-well plates and treated with various concentrations of mouse IFNγ for 72 hours, at which time cells were harvested and counted. IFNγ had significant inhibitory effects on B16F10 melanoma cell growth, as much as 75% inhibition at a concentration of 10 U/ml (FIG. 5). Interestingly, we have found that serum samples from mice given SEA/SEB had IFNγ titers that ranged from 30-300 U/ml. Thus, another possible mechanism for protection from melanoma by superantigen treatment may be the production of cytokines such as IFNγ, which may act directly on the tumor cells to inhibit their growth.

### EXAMPLE 6

### Testing of Superantigens of Varying Vβ Specificities for Enhancing Antigen-Specific Immune Responses

Superantigen compositions are tested following inactivated tumor cell vaccination for the relative ability to protect mice against live tumor challenge. Specifically, SEC1, SEC2, SED, SEE, TSST-1, and MAM and fragments or peptides derived therefrom are tested. C57Bl/6 mice are used, with each treatment group consisting of 8-10 mice. Groups of mice consist of the following:

Group I: No treatment prior to challenge with live tumor cells.

Group II: Treatment consisting of vaccination only.

Group III: Treatment consisting of superantigen only.

Group IV: Treatment consisting of vaccine plus superantigen.

The exact times of superantigen administration and the number of vaccinations are controlled and optimized. Mice are monitored for as long as 90 days in order to determine the relative protective abilities of the superantigen compositions tested. Prior to tumor challenge, mice are monitored for any possible toxic effects of the superantigens. The results obtained in this study form the basis for testing combinations of superantigens in the same animal model. Approximately 300 mice are used for these studies.

### EXAMPLE 7

### Testing of Different Combinations of Superantigens to Expand the Vβ Repertoir Induced against Specific Antigens

To augment the anti-tumor T cell response by increasing the number of activated tumor-specific Vβ T cell subsets in the host, a cocktail of superantigens that activate a wide array of Vβ specific T cells are. Those superantigens that significantly protected mice against lethal tumor challenge will be tested initially in combination with SEA and SEB. The treatment groups will be as follows:

Group I: No treatment prior to challenge with live tumor cells.

Group II: Treatment consisting of vaccination only.

Group III: Treatment consisting of combination of superantigens only.

Group IV: Treatment consisting of vaccine plus combination of superantigens

Mice are monitored for as long as 90 days in order to assess the efficacy of the superantigen combinations. Approximately 200 mice are used for these studies.

### EXAMPLE 8

### Testing of Different Combinations of Superantigens to Expand the Vβ Repertoir Induced against Specific Antigens

In order to determine the mechanism of protection of mice against lethal tumor challenge by vaccination/superantigen treatment, cellular responses and cytokine levels are tested. Specifically, CTL activity of spleen cells against B16F10 melanoma cells. Cytokines are studied by assessing the serum levels of IL-2 and IFNγ as well as the ability of spleen cells from vaccination/superantigen mice to produce IL-2 and IFNγ. These two T cell cytokines are produced in response to superantigen stimulation and are important mediators of immune responses.

These studies are performed initially using SEA and SEB as the prototypic superantigens that confer protection against lethal challenge with live tumor cells. Other superantigens in optimal combinations are tested in a directly analogous manner.

Mice undergo the treatment protocol and are challenged with live tumor cells. Approximately 7-10 days after challenge, mice are sacrificed and spleens are removed. Single cell suspensions of spleen cells are mixed with B16F10 target cells at various effector:target ratios. After overnight incubation, supernatants are assessed for lysis of target cells using the CytoTox 96 Non-Radioactive Cytotoxicity Assay kit (Promega, Madison, Wis.), which measures the presence of lactate dehydrogenase (LDH) in supernatants via a colorimetric assay. LDH is an enzyme that is released upon cell lysis.

Mice are also tested for serum levels of IL-2 and IFNγ utilizing the same mice used for CTL studies. Mice are bled prior to treatment, prior to challenge, and prior to being sacrificed. Serum is tested for the presence of cytokines using commercially available ELISA kits (BioSource International, Camarillo, Calif.). Also, spleen cells are tested for the ability to produce these cytokines in vitro upon superantigen stimulation. Culture supernatants are tested for cytokine production by ELISA. Thus, the in vivo levels of IL-2 and IFNγ, as well as the ability of spleen cells to produce these cytokines in vitro are determined.

Studies such as these help define the parameters involved in protection against lethal doses of melanoma cells.

### EXAMPLE 9

### Vaccination of Humans using Specific Antigens and Superantigens

Based on the above described murine vaccination studies, those skilled in the art will appreciate that humans are vaccinated in analogous fashions, with optimized levels of specific antigens to activate a particular cohort of antigen responsive cells, followed by optimized combinations of superantigens administered at an appropriate time frame in relation to antigen administration to achieve the desired enhancement in the immune response. In the event that anergy is desired, the step of antigen immunization may be eliminated or specific protocols may be implemented to achieve specific anergy of a given cohort of immune responsive cells.

### EXAMPLE 10

### Successful Immunization of Mice against Aggressive Cancer

This invention disclosure demonstrates that we have successfully immunized laboratory mice against melanoma, one of the more aggressive forms of skin cancer. So far, immunized mice have survived for as long as 150 days after exposure to active melanoma cells. Unprotected mice died in a matter of weeks. If we just vaccinate mice with inactivated tumor cells, we get very little protection. But if we vaccinate the mice with inactivated tumor cells and then give them superantigens, we significantly extend the survival of the mice. Superantigens are proteins that are strong stimulators of the immune system. We use the superantigens to boost the response to a vaccine, which in this case was an injection of dead melanoma cancer cells. The research is based on the same process doctors have been using for years to protect people against illnesses such as polio, whooping cough and the flu. The interesting thing about vaccination against infectious diseases is that it's not a miraculous event. What you basically do is inject a part of the harmful organism into an individual under circumstances that will not allow it to grow or cause disease. What you've done is stimulate the immune system of the individual so that it is revved up and is able to kill the infectious agent before it can get a foothold. We have disclosed herein the development of such an approach to dealing with cancers. The problem with cancer is that an individual's immune system doesn't immediately recognize a cancer as something it needs to fight. This isn't an invading bacteria or virus, these are your own cells, and the immune system is primed to not mount an immune response against itself. So when you get an immune response against cancer, part of the problem is that it's foreign and yet it's not, so you get a weaker response. We have demonstrated that by administering an appropriate superantigen at an appropriate dosage and at an appropriate time after administration of the antigen, we are able to amplify the immune response so it becomes a very strong response and can eradicate the tumor. In this manner, the “tug of war” battle between the immune system and a cancer, in which the immune system wants to defend against the cancer but just can't manage it on its own, is tipped on the side of the immune system. Unfortunately in a significant amount of people, the immune system doesn't respond fast enough to be protective against cancer. With superantigens, we tip the scale more in favor of the immune system against this cancer, making it predictable that based on this technology, individuals may be routinely vaccinated against certain cancers. Normally kids are vaccinated against infectious diseases before they attend school. It is not common practice, however, to vaccinate against cancer. But the studies disclosed herein indicate that in cases where tumors have a clear-cut antigen associated with it, we have now provided a methodology for immunization against cancer. It is generally accepted that nothing would please doctors that treat melanoma more than the development of a method of keeping people from coming down with the disease. “Melanoma is a very aggressive form of skin cancer and prevention or early detection are the two keys to its treatment,” said Dr. Robert Skidmore, interim chief of division of dermatology and cutaneous surgery at Shands Hospital at UF. “The possibility of preventing this cancer through immunization would be a fantastic way of reducing both morbidity and mortality associated with melanoma. “It will put me out of business, but that's fine,” he said. “I'll find something else to do.” The American Cancer Society predicts that 47,700 people will be diagnosed with melanoma this year and 7,700 of them will die from the disease.

### EXAMPLE 11

### Protection of Mice against Tumor Challenge is Dependent Timing of Superantigen Administration Following Vaccination

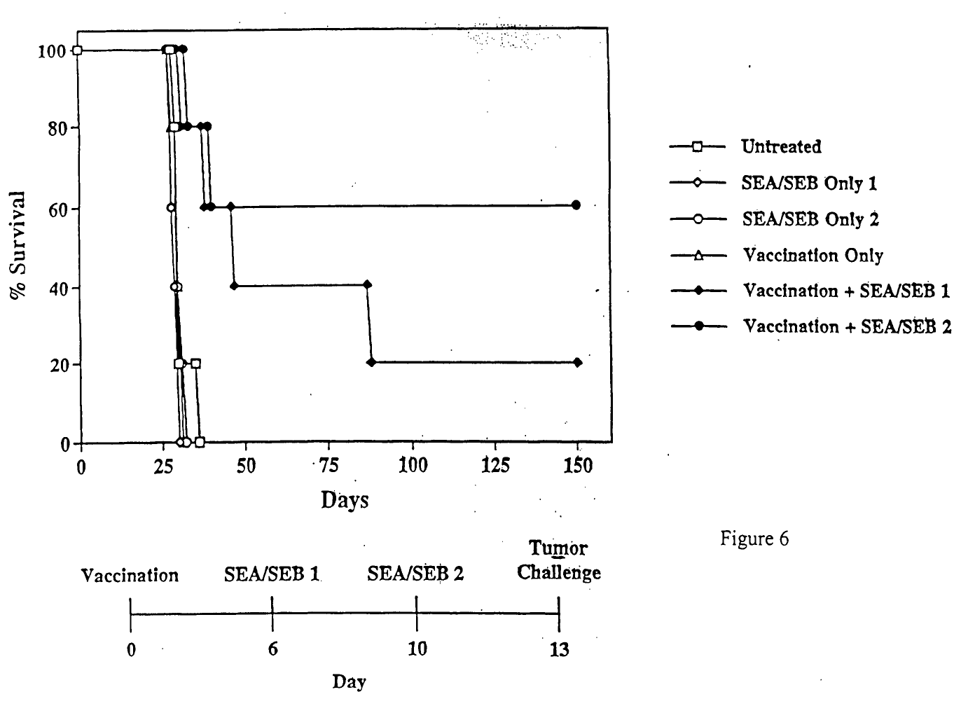

FIG. 6 shows protection of mice against tumor challenge is dependent on the timing of superantigen administration following vaccination. C57BL/6 mice were vaccinated i.p with 1.times.10⁶ irradiated B16F10 melanoma cells at day 0 and subsequently injected i.p. with 25 μg each SEA and SEB at either day 6 or 10. As can be seen, Mice receiving no treatment, vaccination only, and SEA/SEB only served as controls. All mice were challenged i.p. with 2.5×10⁵ live B16F10 cells at day 13. Mice were evaluated on a daily basis and sacrificed when moribund. As can be seen, some mice receiving vaccination and SEA/SEB survived as long as 150 days post challenge, while all control mice died by approximately thirty days post challenge. It is predictable that these results are reproducible in humans. Mice are known to be less receptive to stimulation with SEA and SEB than humans. Accordingly, it is to be anticipated that the human reaction to this treatment is more pronounced. In addition, it is known that SEA is a very potent immune system stimulator. Accordingly, those skilled in the art would understand that SEA alone could be used, SEA plus other superantigens than SEB, or other superantigens, without SEA, through routine experimentation based on this disclosure.

### EXAMPLE 12

### Protection of Mice against Tumor Challenge is Dependent on the Dose of Superantigen Administration Following Vaccination

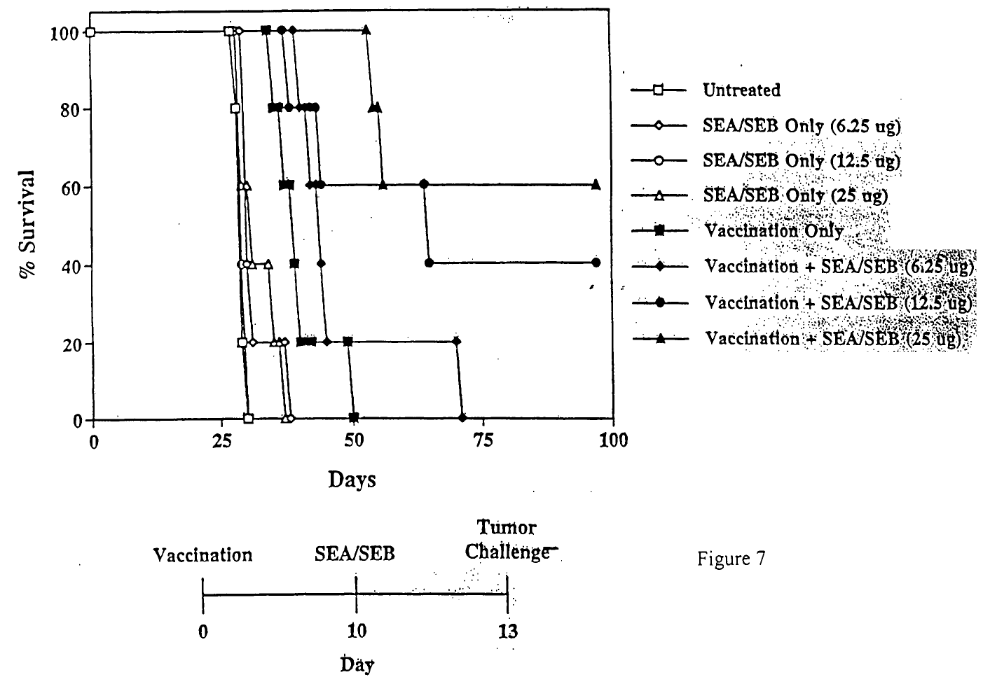

FIG. 7 shows protection of mice against tumor challenge is dependent on dose of superantigen administered following vaccination. C57Bl/6 mice were vaccinated i.p. with 1×10⁶ irradiated B16F10 melanoma cells at day 0 and subsequently injected i.p. with 6.25 μg, 12.5 μg, or 25 μg each SEA and SEB at day 10. As can be seen, Mice receiving no treatment, vaccination only, and SEA/SEB only served as controls. All mice were challenged i.p. with 2.5×10⁵ live B16F10 cells at day 13. Mice were evaluated on a daily basis and sacrificed when moribund. As can be seen, the greater the superantigen dose administered to mice, the greater the survival achieved. Due to possible toxicity side effect, it would be understood that an upper limit in humans would need to be respected in order to avoid such effects. However, those skilled in the art informed with the present disclosure could determine optimal doses through routine, if cautious dose titration in human clinical trials.

### EXAMPLE 13

### Enhancement of Tumoricidal Activity

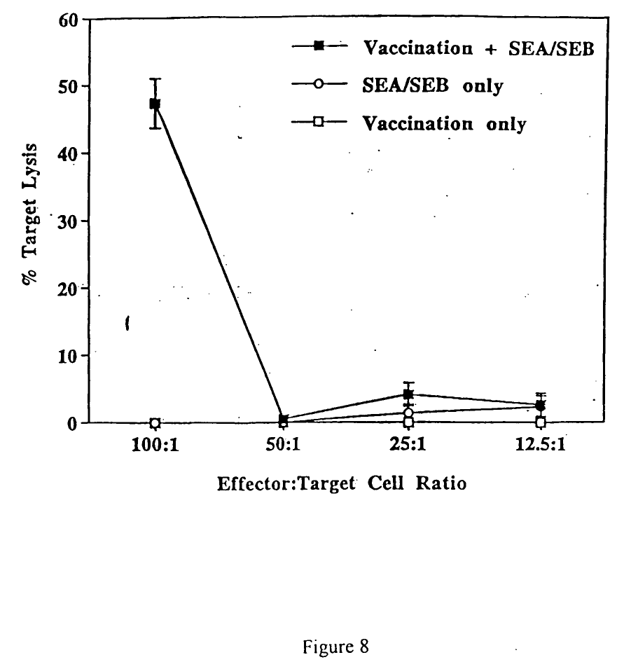

FIG. 8 shows vaccination followed by superantigen adminsitration results in increased tumoricidal activity. C57Bl/6 mice were vaccinated i.p. with 1×10⁶ irradiated B16F10 melanoma cells at day 0 and subsequently injected i.p. with 25 μg each SEA and SEB at day 6. As can be seen, Mice receiving vaccination only, and SEA/SEB only served as controls. Splenocytes were harvested at day 9 and CTL activity was determined by standard 4 hour 51Cr release assay. As can be seen, only splenocytes from mice treated by vaccination and superantigen showed tumoricidal activity at a ratio of 100:1 (effector:target cell ratio).

### EXAMPLE 14

### Superantigen Results in Increaced Cytokine Production

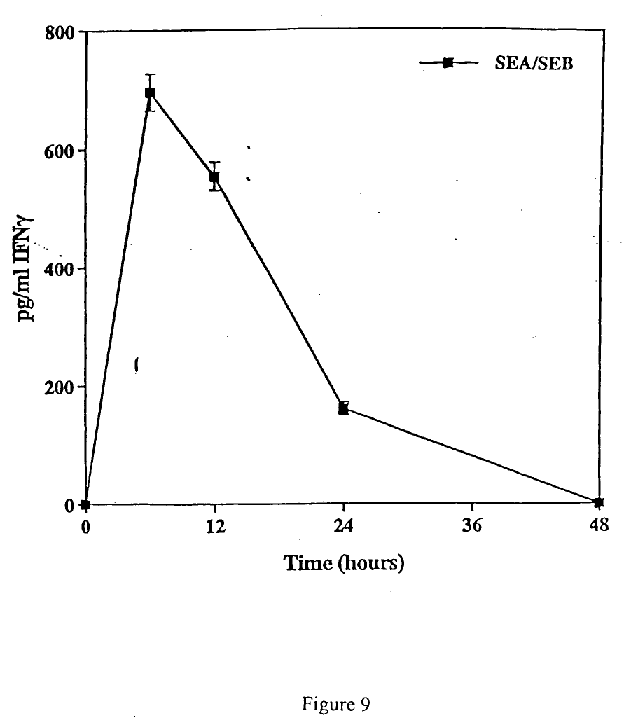

FIG. 9 shows superantigen administration results in increased serum levels of IFNγ. C57BL/6 mice were injected i.p. with 25 μg each SEA and SEB. Serum samples were subsequently collected at appropriate time points and analyzed for IFNγ by ELISA. No IFNγ was detected in serum from mice receiving no treatment. (43 μg is approximately equal to 1 unit IFNγ), while SEA/SEB recipient mice demonstrated a significant increase in cytokine production.

### EXAMPLE 15

### Increased Levels of Cytokines Inhibits Tumor Cell Proliferation

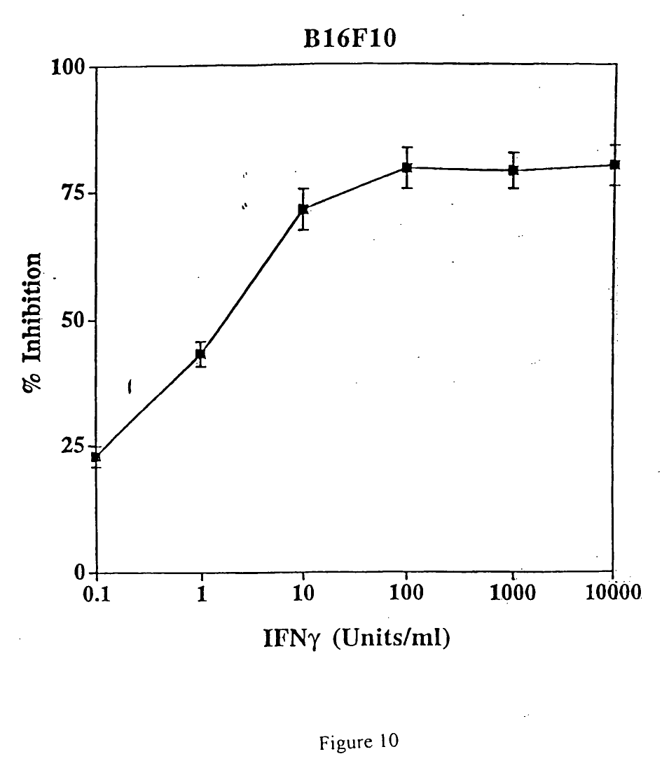

FIG. 10 shows that IFNγ inhibits cellular proliferation of B16F10 melanoma cells in a dose-dependent manner in vitro. Cells (1×10⁴ cells/well) were treated with or without IFNγ at concentrations ranging from 0.1 to 10,000 units/ml for 72 hours. Cells were then harvested and the total number of live cells was determined by direct cell count. Cell viability was approximately 88%. Data from representative experiments are expressed as total number of cells per sample .+-.S.D. Statistical significance was shown by Student's t-test between the number of cells in the presence and absence of IFNγ(P<0.04). It can be seen that increasing levels of cytokine increasingly inhibit cellular proliferation.

### EXAMPLE 16

### Induction of Long-Lasting Immune Memory

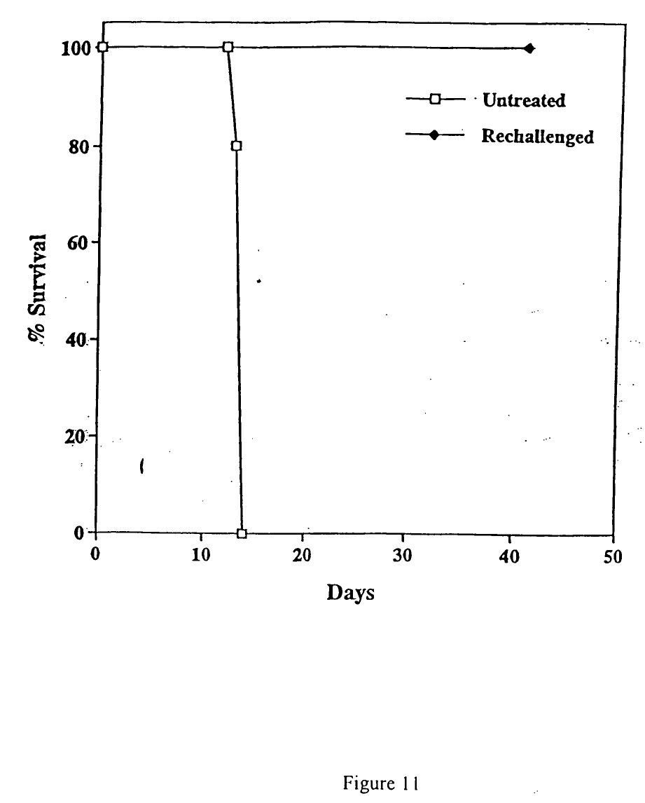

FIG. 11 shows protection of long term survivors against rechallenge with live B16F10 melanoma cells. C57BL/6 mice surviving beyond 150 days following vaccination, superantigen administration, and subsequent live B16F10 tumor challenge were rechallenged i.p with 1×10⁴ live B16F10 cells at day 0. Mice receiving no treatment served as control. Mice were evaluated on a daily basis and sacrificed when moribund. As can be seen, 100% of mice treated by vaccination and superantigen treatment and subsequently challenged, survived rechallenge for up to approximately the 50 day time period followed. This survival of rechallenge demonstrates the elicitation of long-term memory immune responses in the treated animals.

### EXAMPLE 17

### Specificity of the Enhanced Immune Response

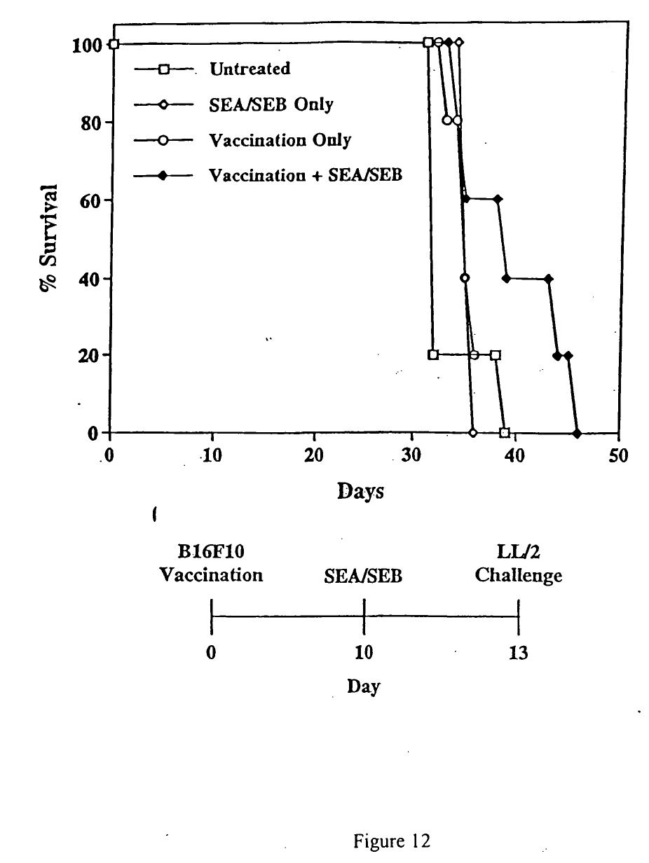

FIG. 12 shows vaccination followed by superantigen administration induces a specific immune response. C57BL/6 mice were vaccinated i.p. with 1×10⁶ irradiated B16F10 melanoma cells at day 0 and subsequently injected i.p. with 25 μg each SEA and SEB at day. 10. Mice receiving no treatment, vaccination only, and SEA/SEB only served as controls. All mice were challenged i.p. with 1×10⁶ live Lewis lung carcinoma (LL/2) cells at day 13. Mice were evaluated on a daily basis and sacrificed when moribund. As can be seen, this treatment does not protect the mice against challenge with an unrelated organism, demonstrating the specificity of the enhanced immune response (i.e. there is not a generalized state of hyper-immunity that is induced).

### EXAMPLE 18

### Method of Treating Disease

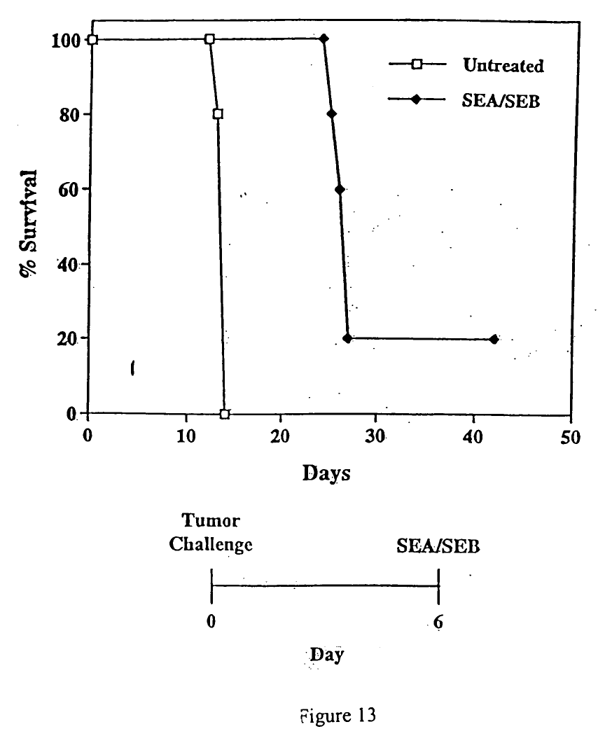

FIG. 13 shows superantigen prolongs the survival of mice with established tumor. C57BL/6 mice were challenged i.p. with live B16F10 cells at day 0 and subsequently injected i.p with 25 μg each SEA and SEB at day 6. Mice receiving no treatment served as control. Mice were evaluated on a daily basis and sacrificed when moribund. Not only does this result demonstrate that animals and humans suffering from a tumor may be directly treated with superantigen and that a beneficial result may be achieved thereby, just as significantly, or more significantly, this result demonstrates that where a tumor is removed, either by excision, radiation treatment, chemotherapy, and the like, subsequent re-exposure of the patient or animal to inactivated tumor cells followed by administration of appropriate superantigen(s) is expected to produce a strong, specific, tumoricidal and anti-tumor immune response. To this end, we have also demonstrated an early NK (nonspecific) immune response, followed by induction of CD4⁺ (antibody inducing) and CD8⁺ (cytolytic) subsequent response.

### REFERENCES

1. Johnson, H. M., B. A. Torres, and J. M. Soos. 1996.) Superantigens: Structure and relevance to human disease. Proc. Soc. Exp. Biol. Med. 212:99-109.

2. Mollick, J. A., R. G. Cook, and R. R. Rich. 1989. Class II MHC molecules are specific receptors for staphylococcal enterotoxin A. Science 244:817-820.

3. D'Orazio, J. A. and J. Stein-Streilein. 1996. Human natural killer (NK) cells present staphylococcal enterotoxin B (SEB) to T lymphocytes. Clin. Exp. Immunol. 104:366-373.

4. Russell, J. K., C. H. Pontzer, and H. M. Johnson. 1990. The I-A.beta.b region (65-85) is a binding site for the superantigen staphylococcal enterotoxin A. Biochem. Biopys. Res. Comm. 168:696-701.

5. Russell, J. K., C. H. Pontzer, and H. M. Johnson. 1991. Both .alpha.-helices along the major histocompatibility complex binding cleft are required for staphylococcal enterotoxin A function. Proc. Natl. Acad. Sci. USA 88:7228-7232.

6. Callahan, J. E., A. Herman, J. Kappler, and P. Marrack. 1990. Stimulation of B110.BR T cells with superantigenic staphylococcal enterotoxins. J. Immunol. 144:2473-2479.

7. Marrack, P. and J. Kappler. 1990. The staphylococcal enterotoxins and their relatives. Science 248:705-711.

8. Peavy, D. L., W. H. Adler, and R. T. Smith. 1970. Mitogenic effects of endotoxin and SEB on mouse spleen cells and human lymphocytes. J. Immunol. 105:1453-1458.

9. Langford, M. P., G. J. Stanton, and H. M. Johnson. 1978. Biological effects of staphylococcal enterotoxin A on human peripheral lymphocytes. Infect. Immun. 22:62-68.

10. Carlsson, R. and H. O. Sjogren. 1985. Kinetics of IL-2 and IFNγ production, expression of IL-2 receptors, and cell proliferation in human mononuclear cells exposed to SEA. Cell. Immunol. 96:175-183.

11. Johnson, H. M. and H. I. Magazine. 1988. Potent mitogenic activity of staphylococcal enterotoxin A requires induction of interleukin 2. Int. Arch. Allergy Appl. Immunol. 87:87-90.

12. Kawabe, Y. and A. Ochi. 1990. Selective anergy of V beta 8⁺CD4⁺ T cells in Staphylococcus enterotoxin B-primed mice. J. Exp. Med. 172:1065-70.

13. Mahlknecht, U., M. Herter, M. K. Hoffmann, D. Niethammer, and G. E. Dannecker. 1996. The toxic shock syndrome toxin-1 induces anergy in human T cells in vivo. Hum. Immunol. 45:42-45.

14. Kotzin, B. L., D. Y. M. Leung, J. Kappler, and P. Marrack. 1993. Superantigens and their potential role in human disease. Adv. Immunol. 54:99-166.

15. Mujtaba, M. G., W. J. Streit, and H. M. Johnson. 1998. IFN-tau suppresses both the autoreactive humoral and cellular immune responses and induces stable remission in mice with chronic experimental allergic encephalomyelitis. Cell. Inmunol. 186:94-102.

16. Tzu, W. and W. R. Fleischmann. 1998. Efficacy of B16 melanoma cells exposed in vitro to long-term IFN.alpha. treatment (B16.alpha. cells) as a tumor vaccine in mice. J. Interferon Cytokine Res. 18:829-839.

17. Hobeika, A. C., W. Etienne, P. Cruz, P. S. Subramaniam, and H. M. Johnson. 1998. IFNγ induction of ^{p21}WAF1 in prostate cancer cells: role in cell cycle, alteration of phenotype and invasive potential. Int. J. Cancer 77:138-145.

18. Kominsky, S. L., H. M. Johnson, G. Bryan, T. Tanabe, A. C. Hobeika, P. S. Subramaniam, and B. A. Torres. 1998. IFN-γ inhibition of cell growth in glioblastomas correlates with increased levels of ^{p21}WAF1/CIP1. Oncogene 17:2973-2979.

19. Hobeika, A. C., W. Etienne, B. A. Torres, H. M. Johnson, and P. S. Subramaniam. 1999. IFN-γ induction of ^{p21}WAF1 is required for cell cycle inhibition and suppression of apoptosis. J. Interferon Cytokine Res. 19:1351-1361.

1. A method for achieving superantigen mediated expansion of antigen-specific T cells for inducing an immune response against melanoma which comprises, prior to melanoma development, administering a melanoma specific antigen composition, followed by administration of a superantigen composition at a optimized time interval following said administering of said melanoma specific antigen composition to enhance the cellular immune response to said antigen, the humoral immune response to said antigen, the cytokine response to administration of said antigen, or combinations of said enhancements, wherein said method enhances resistance against onset of melanoma.

2. The method according to claim 1 wherein said superantigen composition comprises a combined SEA/SEB composition.

3. The method according to claim 1 wherein said superantigen composition is administered at least four days after administration of said antigen.

4. The method according to claim 1 wherein said superantigen composition is administered at least seven days after administration of said antigen.

5. The method according to claim 1 wherein said superantigen composition includes superantigens with Vβ specificities for enhancing antigen-specific immune responses to various pathologic conditions associated with specific antigenic mediators or markers.

6. The method according to claim 1 wherein different combinations of superantigens are administered in order to expand the Vβ repertoire against specific antigens.

7. The method according to claim 1 followed by a regimen of booster vaccinations and superantigen administration at predetermined times and dosages, in relation to the timing and dosage of administering said specific antigens.

8. The method according to claim 1 wherein said superantigen is a superantigen agonist or antagonist peptide or a combination of peptides, peptides and proteins, or combinations of proteins.

9. A method of delaying the onset of tumor development in an animal or human which comprises administering a superantigen at an appropriate time after vaccination with a melanoma specific antigen composition in said animal or human in which said method is practiced.

10. The method according to claim 9 wherein the dosage of superantigen is titrated to achieve the maximum beneficial immune response without inducing unacceptably large toxic side-effects.

11. A method of enhancement of tumoricidal activity which comprises activating splenocytes by treating a human or animal in need of such treatment with a tumor antigen vaccination and subsequently administering one or more superantigens.

12. The method of claim 12, wherein said activating splenocytes by treating a human or animal in need of such treatment with a tumor antigen vaccination and subsequently administering one or more superantigens is followed by a regimen of booster vaccinations and superantigen administration at predetermined times and dosages, in relation to the timing and dosage of administering said specific antigens.

13. The method of claim 12, wherein said tumor antigen vaccination comprises at least one antigen associated with melanoma.

14. The method of claim 12 wherein said subsequently administering on or more superantigens occurs more than seven days after said treating a human or animal in need of such treatment with a tumor antigen vaccination.
